(19) United States
(12) Patent Application Publication
(10) Pub. No.: US 2022/0218519 A1
(43) Pub. Date: Jul. 14, 2022

(54) Appendage Cooling and Heating System and Method of Use

(71) Applicant: SWEETHAWK, LLC., Colleyvile, TX (US)

(72) Inventors: Jana Karika, Colleyville, TX (US); Ken Karika, Colleyville, TX (US)

(73) Assignee: SWEETHAWK, LLC., Colleyvile, TX (US)

(21) Appl. No.: 17/610,560

(22) PCT Filed: May 15, 2020
(86) PCT No.: PCT/US2020/033027

§ 371 Date: Nov. 11, 2021

Related U.S. Application Data
(60) Provisional application No. 62/848,271, filed on May 15, 2019.

(51) Int. Cl. A61F 7/08; A61F 7/10

(52) U.S. Cl. CPC A61F 7/086; A61F 2007/0036; A61F 7/103

(57) ABSTRACT

An appendage cooling and heating system has a body with an outer layer, an inner layer, and an interstitial space for containing a thermo-regulator material. The inner layer and the outer layer are nested to form a joint. An appendage opening is formed in the appendage cooling and heating system to receive an appendage of the user. The joint forms an interlocking seal between the outer layer and the inner layer to contain the thermo-regulator material within the interstitial space.

### 1. FIELD OF THE INVENTION

The present application relates generally to health treatments. In particular, the present application relates to an appendage cooling and heating system for allowing a thermos-regulator, such as ice or hot water, to be removably placed near a hand, foot, or other body areas.

### 2. DESCRIPTION OF RELATED ART

Thermotherapeutical applications and treatments are often recommended as prophylactic measures for those who are experiencing malaise, pain, are ill, or are injured. For example, it is known that heat can increase circulation, bringing an increased flow of blood and nutrients to a specific area of the body. In contrast, cooling treatments slow the blood flow, and may be prescribed to reduce pain and swelling and prevent further injury in athletics, recuperative training, post-cardiovascular procedures, and with homeopathic remedies.

The epidemiology of many injuries, diseases, and illnesses that respond to thermotherapeutical applications can be extremely complex. For example, the epidemiology of nail toxicity and peripheral neuropathy is extremely complex due to the unpredictability and complexity of the nervous system. Although it is known that chemotherapy-induced peripheral neuropathy (CIPN) results from prolonged chemotherapy drugs and systemic treatments, because each patient is unique as well as their reaction to the various types of drugs and treatments, the effective preventative measures prescribed prior to the onset of CIPN are relatively unproven or may vary in intensity, duration, and individual effect.

Certain symptoms are common among athletes, patients, or those experiencing malaise prior to being diagnosed as having a specific illness, injury, nail toxicity, or CIPN. Those common symptoms include tingling sensations, sharp, stabbing pain, throbbing, or burning and/or shock-like sensations. Fingertips, hands, feet, limbs, and joints are generally the affected areas affected.

Prophylaxis and preventative measures for injuries, illnesses, or diseases that respond to thermotherapeutical applications are limited. For example, patients prescribed chemo therapy treatments may receive multiple one- to three-hour intravenous (IV) infusions. Although studies have indicated that thermotherapeutical applications before, during, and/or after these IV infusions may help reduce and/or prevent nail toxicity and minimize the damaging effects of CIPN, the types of applicators and methods of use are often bulky, inefficient, and costly.

The prophylactic and preventative thermotherapeutical applications currently available include submersion in ice tubs or topical application of cold packs, including frozen gel packs, instant cold packs that are crush-activated, and form-fitting freezer gloves and mittens. Gel packs retain their desired temperature for a limited time, and then are unavailable while they are re-frozen. Care facilities must maintain adequate freezer space for each pack per patient for this type of treatment. Crush-activated packs also require additional storage space, and are one-and-done solutions that must be properly disposed of after use. Form-fitting frozen gloves and mittens may cause pain during appendage entry, insertion, and removal, they do not provide adequate room for rotating of appendages or brief removal from the cold surfaces to warmer ambient conditions, and those made with fabric must be sterilized if they are to be re-used by another patient due to the fibers and germ-carrying properties of fabrics.

Although the aforementioned prophylactic and preventative measures represent great strides in the field of thermotherapeutical applications, treatments, and therapy, many shortcomings remain.

Hence, there is a need for a thermotherapeutical application that may increase the effectiveness of preventative measures related to recuperative training, athletic injuries, CIPN, nail toxicity, and other chemo therapy side effects. There is an additional need to reduce therapy complications, cost, waste, and storage requirements of current solutions and prophylactic measures in the field of thermotherapeutical applications.

### DESCRIPTION OF THE DRAWINGS

The novel features believed characteristic of the invention are set forth in the appended claims. However, the invention itself, as well as a preferred mode of use, and further objectives and advantages thereof, will best be understood by reference to the following detailed description when read in conjunction with the accompanying drawings, wherein:

FIG. 1 is a front elevational view of a preferred embodiment of an appendage cooling and heating system, according to the present application;

FIG. 2 is a front elevational view of a joint of the appendage cooling and heating system of FIG. 1;

FIG. 3 is a top view of a ring for forming the joint of FIG. 2;

FIG. 4 is an alternative embodiment of a joint of an appendage cooling and heating system, according to the present application;

FIG. 5A is front elevation view of an alternative embodiment of an appendage cooling and heating system, according to the present application;

FIG. 5B is a top view of the appendage cooling and heating system of FIG. 5A;

FIG. 5C is a section view of the appendage cooling and heating system of FIG. 5A taken along Section Line 5C;

FIG. 5D is an exploded view of a joint of the appendage cooling and heating system of FIG. 5A;

FIG. 6 is an assembly view of an alternative embodiment of an appendage cooling and heating system, according to the present application;

FIG. 7 is a cross-sectional top view of an alternative embodiment of an appendage cooling and heating system, according to the present application;

FIG. 8 is a cross-sectional top view of an alternative embodiment of an appendage cooling and heating system, according to the present application; and

FIG. 9 is a flowchart of a preferred method of use, according to the present application.

### DETAILED DESCRIPTION OF THE PREFERRED EMBODIMENT

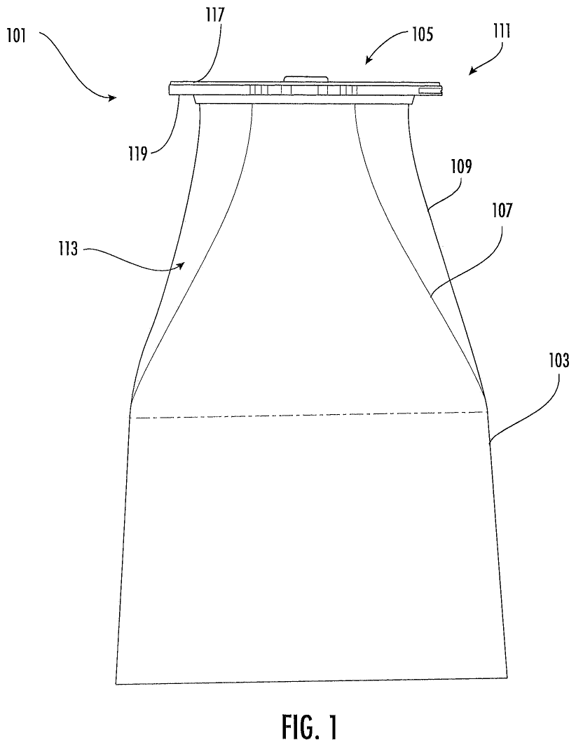

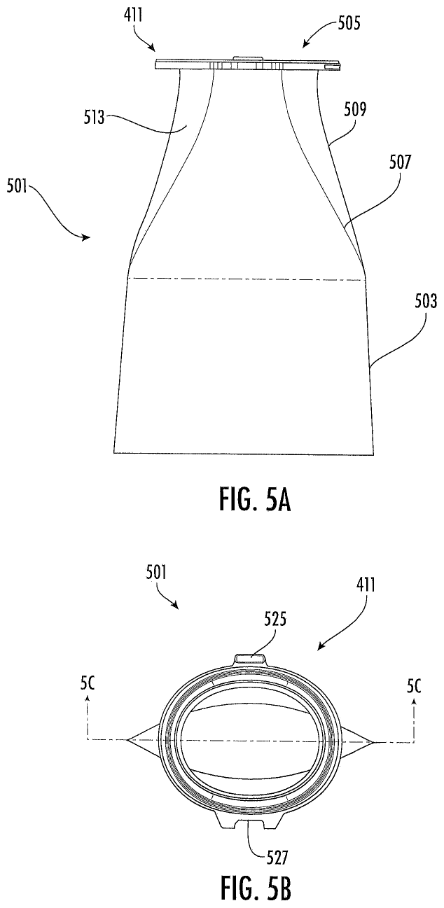

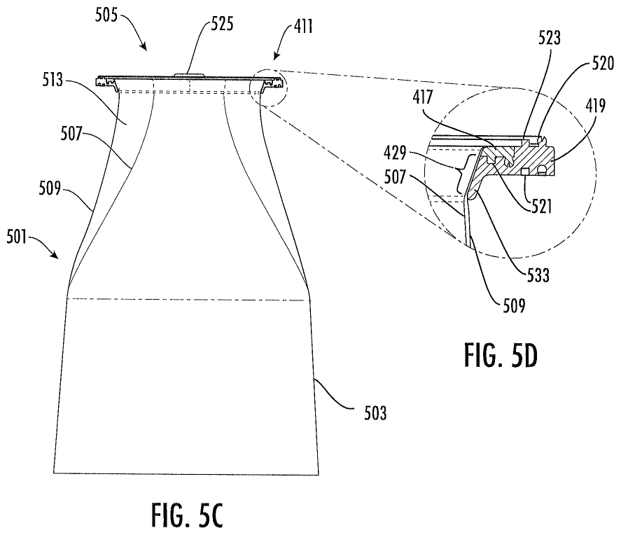

Referring now to FIG. 1 in the drawings wherein like reference characters identify corresponding or similar elements throughout the several views, an appendage cooling and heating system 101 is illustrated. Cooling system 101 includes a body 103, an appendage opening 105 for receiving an appendage, such as a hand, foot, finger, or limb. The body 103 includes an inner layer 107 and an outer layer 109, which are connected together by a joint 111. Between the inner layer 107 and the outer layer 109 is an interstitial space 113 for containing a reusable, cost-effective heat sink medium (see FIG. 5, below), such as ice.

It is important to note that ice machines are usually found on the premises of care facilities for a wide variety of purposes. Large quantities of ice can be produced by a single ice machine. Melted ice is easily disposed of, environmentally friendly, and easily exchanged when temperatures are no longer adequate to produce the desired effect.

In a preferred embodiment, the appendage opening 105 is elongated or elliptical in shape. For example, the major axis of the opening may be approximately 10-15% longer than the minor axis of the opening.

In a preferred embodiment, the joint 111 is made from an upper ring 117 nested within a lower ring 119. In a preferred embodiment, the upper ring 117 and the lower ring 119 are identical in shape and design, just rotated, such that one ring fits on top of the and partially within the other. For example, the upper ring 117 has protruding portions inserted in corresponding recessed portions of the other. In a preferred embodiment, the upper and lower rings 117, 119 snap together. Alternatively, the rings 117, 119 are connected together to form joint 111 by threaded attachment, clasps, tabs, interlocking flanges, and combinations thereof.

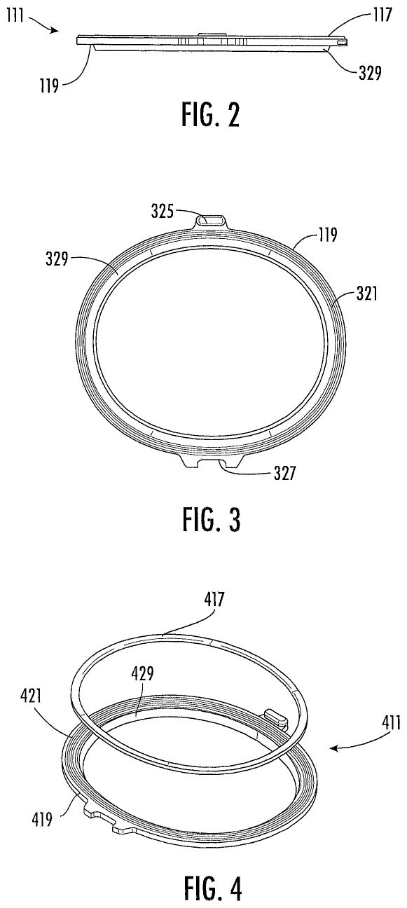

Referring now to FIG. 2 in the drawings, a side elevation view of the joint 111 is illustrated. Although not shown in FIG. 2, the inner and outer layers 107, 109 are adhered to portions of the upper and lower rings 117, 119. Preferably, a heat fusion weld or an ultrasonic welding process is used to adhere the layers to at least one of a trough, a ridge, or a cuff of the respective ring 117 or 119.

Referring now to FIG. 3 in the drawings, in a preferred embodiment, the lower ring 119 includes at least one annular trough 321 located on a top side of the ring. The annular trough 321 is collinearly aligned with at least one annular ridge (see FIG. 5C, below) located on an underside of the ring. Preferably, the ring 119 includes multiple troughs 321 on one side and multiple ridges on the opposite side with two or more ridges and troughs that are collinearly aligned in each side. The shape of the troughs and ridges may vary, including circular, elliptical, and square shaped designs. In general, the shapes of the troughs and ridges are similar in shape to the ring itself, and are therefore preferably elliptically shaped. The shape of the sidewalls of the troughs and ridges may vary, including bulbous, rounded, straight-edged, and angled shapes. Preferably, at least one annular ridge has a bulbous shape that includes a curved head portion, a transition portion, and a narrow neck portion is used. This ensures a primary interlock when the at least one annular ridge is inserted into a correspondingly shaped, annular, collinear trough.

In a preferred embodiment, a trough 321 has a slightly larger dimension than a corresponding, collinear ridge. For example, a trough may have a width that is 8-12% wider than a width of the corresponding, collinear ridge.

In at least one embodiment, a trough 321 is configured to receive another ring to create a seal for the joint 111. For example, the trough 321 may be sized to receive an O-ring or a gasket within the trough, such that pressure between it, a surface of a ring 117 or 119, and at least one of the layers 107, 109 creates the seal for the joint 111.

In a preferred embodiment, the rings 117, 119 are made of a plastic material, such as polyethylene, which has a similar or identical melting point as the material used to form the inner and outer layers 107, 109. Preferably, the material used for the layers 107, 109 and the rings 117, 119 are identical in composition, such that both are made from a single, water-tight material, such as polyethylene or polypropylene, but with different material thicknesses. Alternatively, other plastics, elastomers, material blends, or moldable and/or extruded materials may be used such as polytetrafluoroethylene (Teflon or PTFE), silicon, and pliable polymer-dipped metal/wire frame materials. It is noted that when the alternative materials are used, an adhesive may be used instead of a heat/ultrasonic weld to adhere the inner and outer layers 107, 109 to the upper and lower rings 117, 119. In at least one embodiment, both an adhesive and a heat/ultrasonic weld are used. The adhesive includes an environmentally friendly polymer adhesive, such as poly(propylene-co-glycidyl butyrate carbonate) or PPGBC.

In a preferred embodiment, the ring 119 includes a protruding tab 325 opposite a recess 327 in the ring 119. Preferably, both the tab 325 and the recess 327 are formed in the unitary structure of the ring 119. The tab 325 and the recess 327 of a first ring correspondingly fit within the tab and recess of a second ring. For example, once the second ring is rotated and oppositely oriented, the tab 325 fits within recess 327. This provides a secondary interlock to the rings of the joint 111 and also provides a user-friendly separation mechanism.

In at least one embodiment, tab 325 has both a raised, keyed portion on a top side of the tab and a recessed receiving portion on its underside, such that the raised, keyed portion of another tab of a second ring fits within the recessed portion on the underside of the first ring. The the raised key-like portion extends above the remainder of the surface of tab 325. Although the raised portion is depicted as being located on a top side of the tab 325, alternatively the tab 325 may be configured to have a recessed portion located on the top side of the tab 325, where the recessed portion receives a raised portion of a tab of a second ring. In these embodiments, the recess 327 may be optional, as the ring 119 could have two tabs positioned opposite each other, or multiple tabs positioned around a circumference of the ring 119.

In at least one embodiment, the shape of the lower ring 119 is elliptical, having a substantially flat top surface where the only sloping parts are located in the troughs and ridges of the ring. Alternatively, the top surface of the lower ring 119 slopes concentrically inward, creating a coned-disc shape, or a conical spring washer shape similar to a Belleville washer.

In at least one embodiment, the ring 119 is formed with a cuff 329 extending out and away from the top surface. Preferably, the cuff 329 is formed concentrically sloping inward at a different angle than the slope of the top surface of the ring 119. For example, the cuff 329 is formed at a 90° angle relative to a plane parallel to the top surface, By way of another example, the cuff 329 is formed at a 10°, 15°, 20°, 25°, or 30° angle relative to a plane parallel to the top surface.

Preferably, the cuff 329 interacts with a layer 107 or 109 adhered within the annular trough 321. For example, the outer layer 109 may be adhered both within the annular trough 321 and to at least a portion of an intrados surface of the cuff 329, In at least one embodiment, the inner layer 107 is inserted through an appendage opening and is wrapped completely around an O-ring that fits within the lower ring 119, such that a portion of the material making up the inner layer 107 is folded onto itself around the O-ring.

In at least one embodiment, a single sheet of layer 107 or 109 is adhered to the lower ring 119 without any sublayers or overlaps. Alternatively, an end of the single sheet of material making up the outer layer 109 is rolled and/or folded onto itself to form multiple sub-layers, and the multiple sub-layers created by the rolling and/or folding are adhered within the trough 321.

Referring now to FIG. 4 in the drawings, an assembly view of an alternative embodiment of a joint 411 is illustrated. The alternative joint 411 is formed of two sets of rings, where a ring in each set is different from another ring in the set relative to at least one of: 1) material composition, 2) shape, 3) size, 4) dimension, and 5) number of corresponding, collinear troughs and/or ridges. An interlocking ring 419 of the joint 411 includes a series 421 of troughs and ridges. A bag-holding ring 417 is nested within the innermost circumference of the interlocking ring 419. The series 421 of troughs and ridges of the interlocking ring 419 mate with another series of troughs and ridges of an interlocking ring of the second set of rings. Thus, two sets of interlocking rings 419 and bag-holding rings 417 form the joint 411. The seal for joint 411 in this embodiment is created by pressure and tension. For example, either the cuff 429 of the lower ring 419 tapers, or the nested ring 417 tapers, such that the ring 417 and its attached inner layer 107 are wedged inside the cuff 429. The tension from being wedged creates the seal for the joint 411.

In an alternative embodiment, both rings 417 and 419 are bag-holding rings, and joint 411 is formed by inserting ring 417 within a portion of the ring 419, and this insertion alone creates the seal for joint 411. For example, the lower ring may include four or more troughs and ridges in the series 421, and the nested ring 417 has only half as many correspondingly shaped, collinear troughs and ridges. The inner layer 407 is wrapped through the opening in ring 417 and around each of its troughs and ridges. These troughs and ridges having material wrapped around them snap into and between at least two of troughs and ridges of the series 421 of troughs and/or ridges of the lower ring 419. The extra troughs and ridges of the series 421, i.e., that are not used to receive inner layer 407 and ring 417, may optionally be used with a second set of rings and a second outer layer, such that the body of the system has at least three layers.

In a preferred embodiment, the sidewalls of the troughs and ridges of the series 421 are slightly angled based on a desired interaction with another ring having a second series of troughs and ridges. For example, an inner ridge is angled inward or towards the inserted appendage, while the outermost ridge, i.e., ridge with largest circumference, is angled outward or away from the inserted appendage. Corresponding troughs of a second or a third ring may be similarly angled to increase tension between sidewalls of troughs and/or ridges and thereby increase the effectiveness of the seal.

The nested elliptically shaped ring 417 may be rigid, semi-rigid, or flexible. The ring 417 has a circumference that is slightly larger than the inner circumference of the lower ring 419, further enabling the seal of joint 411.

Referring now to FIGS. 5A through 5D in the drawings, an alternative embodiment of an appendage cooling and heating system 501 having joint 411 is illustrated. Cooling system 501 includes a body 503, an appendage opening 505 for receiving an appendage, such as a hand, foot, finger, or limb. The body 503 includes an inner layer 507 and an outer layer 509, which are connected together by the joint 411. Between the inner layer 507 and the outer layer 509 is an interstitial space 513. Although the layers 507, 509 are preferably adhered to an innermost trough 521, alternatively the layers 507, 509 are adhered within any of the troughs 521 or onto any of the ridges 523 of the series 421 of annular troughs and ridges.

Referring specifically now to FIG. 5D in the drawings, an exploded view of the joint 411 is illustrated. In a preferred embodiment, the joint 411 is made from an inner/upper ring 417 nested within a lower ring 419, where the lower ring has at least one annular trough 521 and a correspondingly shaped, collinear annular ridge 523.

A rounded tab 533 is formed at the end of the cuff 429. The outer layer 509 of system 501 is attached to at least one of an annular trough 521, the rounded tab 533, and the cuff 429. In at least one embodiment, two identically shaped upper and lower rings 419 are snapped together, and the rounded tab 533 of the respective upper and lower rings provide a second location at which a user may separate the rings from each other—the first location being the tab 525 and recess 527.

In the appendage cooling and heating system 501, preferably, at least one of the annular ridges 523 has a groove or a notch 520 cut or otherwise formed therein. For example, the groove or notch 520 may have angled sides that come to a point at one end of the ridge 523, and be open and spaced apart at the other end. The sides of the ridge 523 receive pressure from inserting the ridge into a corresponding trough of another ring and the groove or notch 520 provides room to contract the sides of the ridge 523.

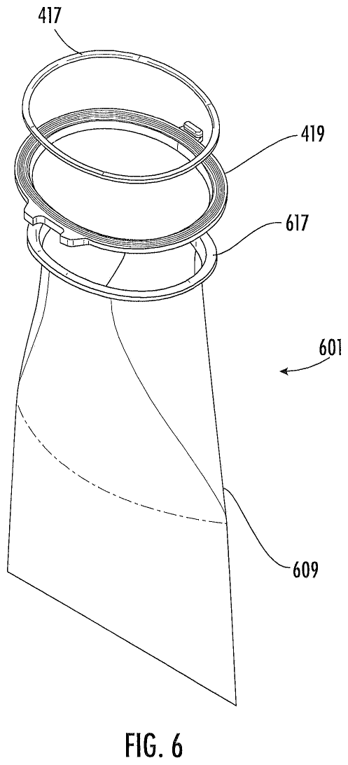

Referring now to FIG. 6 in the drawings, an assembly view of an alternative embodiment of an appendage cooling and heating system 601 using joint 411 and using a third ring 617 is illustrated. The appendage cooling and heating system 601 uses two rings to form a joint 411 similar to the joint depicted in FIG. 5D. In a preferred embodiment, only the ring 419 and the ring 417 have bags, or respective inner and outer layers 507, 509 attached to them. Alternatively, each of the rings 417 and 419 have respective inner layers attached to the respective rings, while the third ring 617 has outer layer 609 attached to the third ring 617. In this embodiment, various combinations of snapping, wedging, and nesting of rings are obtainable depending on the shape, size, dimension, and trough/ridge configuration of the respective rings. For example, two rings may be nested within a third ring, snapping troughs and ridges of the two rings to correspondingly shaped troughs and ridges of the third ring. By way of another example, one ring may be nested within a second ring, while the third ring is used as a wedge ring to wedge inside the inner circumferences of the other two rings. Multiple different combinations and variations will be recognized by those skilled in the art, and each of the various combinations are encompassed by the present application.

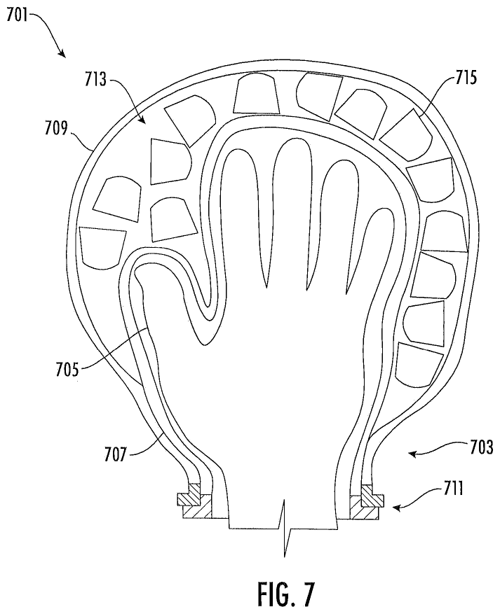

Referring now to FIG. 7 in the drawings, an alternative embodiment of an appendage cooling and heating system 701 is illustrated. System 701 includes a body 703 and an appendage opening 705 that is configured to fit over and around an appendage. The body 703 includes an inner layer 707 removably attached to an outer layer 709 via a joint 711. Between the inner layer 707 and the outer layer 709 exists an interstitial space 713, wherein a thermo-regulator 715 is held. Although the interstitial space 713 appears to be in the form of a mitten, or generally in the shape of a hand, preferably seams and/or fold lines are removed such that the inner layer is not formed into the shape of a mitten. However, in some embodiments, a sleeve, tube, or sock-like shape is used. These shapes may be conducive to a leg, arm, foot, and/or hand without restricting movement within the body 703.

In use, the outer layer 709 is removed via a separation of the joint 711 in order to place the thermo-regulator material 715 within the interstitial space 713. Using the appendage opening 705, the inner layer 707 is placed over the appendage of the user. The outer layer 709 is filled with the thermo-regulator material 715, and then placed over the inner layer 707 and secured at the joint 711.

Preferably, the components of the joint 711 snap together. Alternatively the joint 711 may comprise a twist lock, fastener, or another method of sealing the interstitial space 713, making it water-tight.

It is important to note that once the temperature differential between the heat sink 715 and the appendage or the ambient air has equilibrated or is nullified, the inner layer 707 remains intact and does not need to be removed from the appendage or its ring in order to replace the thermo-regulator material. Only the outer layer and ring need be removed in order to replace or refill the thermo-regulator material.

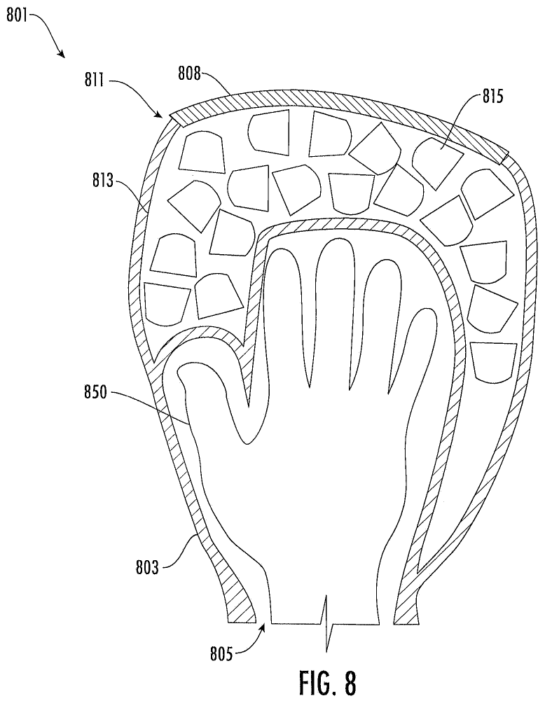

Referring now to FIG. 8 in the drawings, an alternative embodiment of an appendage cooling and heating system 801 is illustrated. Cooling system 801 includes a body 803 and an appendage opening 805. An access point 808 having a threaded joint 811 is disposed at an end opposite the appendage opening 805. The access point 808, such as a threaded plug, may be removed to access interstitial compartment 813, where the thermo-regulator material 815 is stored adjacent a compartment attached to appendage opening 805 in which an appendage 850 is inserted.

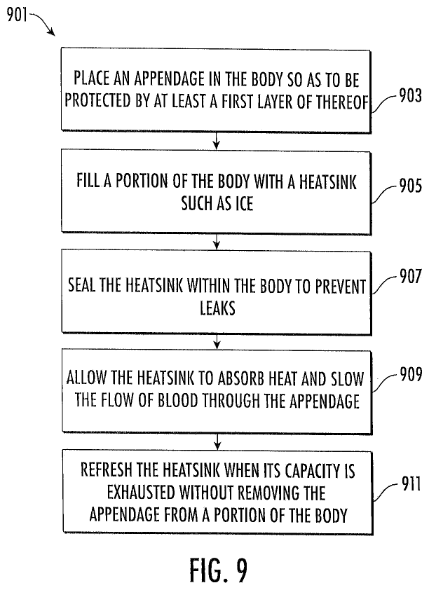

Referring now to FIG. 9 in the drawings, a method 901 of cooling an appendage, is illustrated.

Step 903 includes placing an appendage in the body of an appendage cooling and heating system so as to protect the appendage by a first layer of the cooling system. For example, a hand is inserted into appendage opening 105 inside inner layer 107.

Step 905 includes adding a thermo-regulator material to a portion of the body of the appendage cooling and heating system. For example, the outer layer 109 is partially filled with a heatsink, such as ice and/or cold water, or with a heat source, such as hot water.

Step 907 includes sealing or interlocking rings of the body of the appendage cooling and heating system such that the thermo-regulator material is contained without leaking. For example, an inner ring 117 may interlock with a lower ring 119, with the pressure of cuffs and/or angled interlocking troughs and ridges providing a water-tight seal at joint 111.

Step 909 includes facilitating the absorption, or equilibrating, of a heat differential from the body of the appendage cooling and heating system into the thermo-regulator material. For example, although thin fabric gloves may be worn by the hand of the user that is inserted into appendage opening 105, the user is careful that thick fabric or excessive insulation is not applied to the appendage within appendage opening 105.

Step 911 includes refreshing the thermo-regulator material when the heat differential between the thermo-regulator and the appendage or the ambient air within the body of the cooling system has equilibrated or been nullified. For example, the capacity of the ice 815 to cool the appendage may be exhausted when the ice 815 has completely melted. At this point, the user may or may not remove their hand from the body 803 of cooling system 801 as the interstitial compartment 813 is refilled with ice 815, and the body is resealed. In at least one embodiment, leaving the hand in the body 803 while refreshing the ice is optional.

It is noted that methods of manufacture may include extrusion techniques, extension of materials onto a mandrel to form a material such as for a bag/layer, reverse vacuum and/or heat sealing to attach a bag/layer to a ring, dissolving or collapsing the mandrel to remove the mandrel from the formed materials, folding or rolling the formed materials, vacuum sealing for storage, packing, and shipment, and other similar processes. The order and exact recitation of these steps is not meant to be limiting, as those skilled in the art will recognize reordering, modifications, and/or combinations of these steps, each of which are intended to be encompassed by the present application.

It is apparent that an invention with significant advantages has been described and illustrated. Although the present application is shown in a limited number of forms, it is not limited to just these forms, but is amenable to various changes and modifications without departing from the spirit thereof.

1. An appendage cooling and heating system, comprising:
a body, comprising:
a non-rigid outer layer;
an inner layer; and
an interstitial space for containing a thermo-regulator material;
a rigid joint; and
an appendage opening;
wherein the appendage opening is configured to receive an appendage of a user; and
wherein the joint forms an interlocking seal between the outer layer and the inner layer to contain the thermo-regulator material within the interstitial space; and
wherein the interstitial space is continuous relative to both the outer layer and the inner layer.

2. The appendage cooling and heating system according to claim 1, wherein the joint comprises:
a ring having an annular trough and an annular ridge.

3. The appendage cooling and heating system according to claim 2, further comprising:
a second ring.

4. The appendage cooling and heating system according to claim 3, wherein at least one of the second ring, the annular trough, and the annular ridge has a dimension that is larger than a corresponding ring, annular trough, or annular ridge.

5. The appendage cooling and heating system according to claim 2, wherein the annular trough and the annular ridge are collinearly aligned.

6. The appendage cooling and heating system according to claim 2, further comprising:
a first series of vertically-oriented annular troughs and ridges.

7. The appendage cooling and heating system according to claim 6, further comprising:
a second series of vertically-oriented annular troughs and ridges;
wherein at least one of the annular ridges of the first series of annular troughs and ridges are angled to interlock with at least one angled trough of the second series of annular troughs and ridges.

8. The appendage cooling and heating system according to claim 1, wherein the thermo-regulator comprises a heatsink.

9. A ring for an appendage cooling and heating system, comprising:
a cuff;
a tab;
a rigid first annular trough for adhering a water-tight layer of the appendage cooling and heating system;
a rigid second annular trough for receiving a rigid annular ridge of a second ring of the appendage cooling and heating system; and
a rigid first annular ridge;
wherein the rigid first annular trough and the rigid first annular ridge are collinearly aligned; and
wherein the rigid first annular trough, the rigid second annular trough, and the rigid first annular ridge are vertically-oriented.

10. The ring for an appendage cooling and heating system according to claim 9, wherein the second annular trough has a dimension that is larger than a corresponding dimension of the annular ridge of the second ring of the appendage cooling and heating system.

11. The ring for an appendage cooling and heating system according to claim 10, wherein the second annular trough and the annular ridge of the second ring of the appendage cooling and heating system fit together to form a joint.

12. The ring of the appendage cooling and heating system according to claim 9, further comprising:
a third vertically-oriented annular trough; and
a second vertically-oriented annular ridge.

13. The ring of the appendage cooling and heating system according to claim 12, wherein the third vertically-oriented annular trough and the second vertically-oriented annular ridge are collinearly aligned.

14. The ring of the appendage cooling and heating system according to claim 13, wherein the third vertically-oriented annular trough has a dimension that is larger than a corresponding dimension of the second vertically-oriented annular ridge.

15. The ring of the appendage cooling and heating system according to claim 14, wherein the third vertically-oriented annular trough and a second vertically-oriented annular ridge of the second ring of the appendage cooling and heating system fit together to form a water-tight joint.

16. The ring of the appendage cooling and heating system according to claim 9, further comprising:
a series of vertically-oriented annular troughs and ridges.

17. The ring of the appendage cooling and heating system according to claim 16, wherein at least one of the annular ridges of the series of vertically-oriented annular troughs and ridges are angled to interlock with at least one angled trough of a series of vertically-oriented annular troughs and ridges of the second ring of the appendage cooling and heating system.

18. A method for cooling an appendage, comprising:
providing a body of an appendage cooling and heating system, having:
a non-rigid outer layer;
an inner layer; and
an interstitial space for containing a thermo-regulator material, the interstitial space being continuous relative to both the outer layer and the inner layer;
providing an appendage opening in the appendage cooling and heating system;
wherein the appendage opening is sized to receive an appendage of a user; and
placing the appendage within the inner layer of the body;
adding the thermo-regulator material within the outer layer of the body;
nesting the inner layer together with the outer layer to form a sealed joint; and
wherein the sealed joint forms an interlocking seal between the outer layer and the inner layer to contain the thermo-regulator material within the interstitial space.

19. The method according to claim 18, further comprising:
facilitating energy absorption from the thermo-regulator material to the appendage.

20. The method according to claim 19, further comprising:
repeating the step of adding the thermo-regulator material to the outer layer of the body.

21. The method according to claim 20, wherein the thermo-regulator material is added without removing the appendage from the body.
